(19) United States
(12) Patent Application Publication
(10) Pub. No.: US 2022/0383466 A1
(43) Pub. Date: Dec. 1, 2022

(54) IMAGE PROCESSING APPARATUS, IMAGE PROCESSING METHOD, AND STORAGE MEDIUM

(71) Applicant: CANON KABUSHIKI KAISHA, Tokyo (JP)

(72) Inventors: Akira Tsukuda, Tokyo (JP); Takeshi Noda, Kanagawa (JP); Atsushi Iwashita, Tokyo (JP); Masayoshi Tokumoto, Kanagawa (JP)

(21) Appl. No.: 17/878,965

(22) Filed: Aug. 2, 2022

Related U.S. Application Data
(63) Continuation of application No. PCT/JP2021/003698, filed on Feb. 2, 2021.

(30) Foreign Application Priority Data
Feb. 19, 2020 (JP) 2020-026470

(51) Int. Cl. G06T 5/50

(52) U.S. Cl. CPC G06T 5/50; G06T 2207/10116; G06T 2207/20224; G06T 2207/30021

(57) ABSTRACT

An image processing apparatus that processes a radiation image generates a decomposition image representing a planar distribution related to a material, using a plurality of radiation images of an object containing a target object that correspond to different radiation energies, and obtains a target object image related to the target object using a band limitation image corresponding to a frequency band related to a size of the target object, the band limitation image being obtained by performing frequency decomposition on the decomposition image.

### CROSS-REFERENCE TO RELATED APPLICATIONS

This application is a Continuation of International Patent Application No. PCT/JP2021/003698, filed Feb. 2, 2021, which claims the benefit of Japanese Patent Application No. 2020-026470, filed Feb. 19, 2020, both of which are hereby incorporated by reference herein in their entirety.

### BACKGROUND OF THE INVENTION

### Field of the Invention

The present invention relates to an image processing apparatus, an image processing method, and a storage medium.

### Background Art

In recent years, spectral imaging technology, which is an imaging technology that utilizes radiation energy information, has been widely studied or put to practical use as one of the technical fields of radiation imaging apparatuses. Spectral imaging can be used to obtain a thickness distribution of a plurality of materials from multiple energy images, or to obtain an area density distribution and an effective atomic number distribution. The thickness distribution, area density distribution, and effective atomic number distribution are examples of planar distributions related to materials. Such planar distributions can be obtained by backward calculation from pixel values of transmitted X-rays, while assuming that the radiation after transmission through the material attenuates as an exponential function of the thickness or the like.

In spectral imaging, images shot with two different levels of energy are often used to obtain planar distributions related to two materials; e.g., a bone thickness distribution and a soft tissue thickness distribution. Here, if the human body contains materials other than bones and soft tissues, e.g., if a contrast agent is present in the image in addition to bones and soft tissues, the image of the contrast agent is included in the images of the other materials (e.g., a bone image) and cannot therefore be well separated. One suggested method for separating bone and a contrast agent is to perform radiographic imaging twice using high-level energy and low-level energy for the state without the contrast agent and the state therewith, and extract the contrast agent based on the difference between the two bone images obtained (PTL 1).

However, when the technique in PTL 1 is used, the number of times of imaging increases because imaging in the state without a contrast agent is required to generate a mask image in order to extract a contrast agent image.

The present invention provides a technique that enables materials to be separated from a decomposition image obtained by means of spectral imaging or the like, without using a mask image.

### CITATION LIST

### Patent Literature

PTL1: Japanese Patent Laid-Open No. S58-221580

PTL2: Japanese Patent Laid-Open No. 2018-511443 (which will be referenced in the embodiments)

### SUMMARY OF THE INVENTION

According to one aspect of the present invention, there is provided an image processing apparatus that processes a radiation image comprising: a generation unit configured to generate a decomposition image representing a planar distribution related to a material, using a plurality of radiation images of an object containing a target object that correspond to different radiation energies; and an obtaining unit configured to obtain a target object image related to the target object using a band limitation image corresponding to a frequency band related to a size of the target object, the band limitation image being obtained by performing frequency decomposition on the decomposition image.

According to another aspect of the present invention, there is provided an image processing method for processing a radiation image comprising: generating a decomposition image representing a planar distribution related to a material, using a plurality of radiation images of an object containing a target object that correspond to different radiation energies; and obtaining a target object image related to the target object using a band limitation image corresponding to a frequency band related to a size of the target object, the band limitation image being obtained by performing frequency decomposition on the decomposition image.

According to another aspect of the present invention, there is provided a non-transitory computer-readable storage medium storing a program for causing a computer to execute the above-described image processing method.

Further features of the present invention will become apparent from the following description of exemplary embodiments with reference to the attached drawings.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 shows an example configuration of an X-ray imaging apparatus according to a first embodiment.

FIG. 2 is a flowchart showing imaging processing according to the first embodiment.

FIG. 3A shows an example of three-material decomposition processing according to the first embodiment.

FIG. 3B shows an example of three-material decomposition processing according to the first embodiment.

FIG. 4 illustrates an example of a method for specifying a target object frequency.

FIG. 5 shows an example of frequency decomposition processing.

FIG. 6 shows an example of a method for combining band limitation images according to a second embodiment.

FIG. 7 shows an example of a method for setting a weighting coefficient.

FIG. 8A shows an example of four-material decomposition processing according to a third embodiment.

FIG. 8B shows an example of four-material decomposition processing according to the third embodiment.

FIG. 9 is a flowchart showing imaging processing according to a fourth embodiment.

FIG. 10 shows an example of four-material decomposition processing according to the fourth embodiment.

FIG. 11 shows another example of four-material decomposition processing according to the fourth embodiment.

FIG. 12 illustrates improvement of decomposition accuracy with use of auxiliary information according to a fifth embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

Note that an example of using X-rays as radiation will be described below, but radiation in the present invention is not limited to X-rays. Radiation may include not only α-rays, β-rays, and γ-rays, which are beams created by particles (including photons) emitted due to radiation decay, but also beams with similar or higher-level energy, such as X-rays, particle rays, and cosmic rays.

### First Embodiment

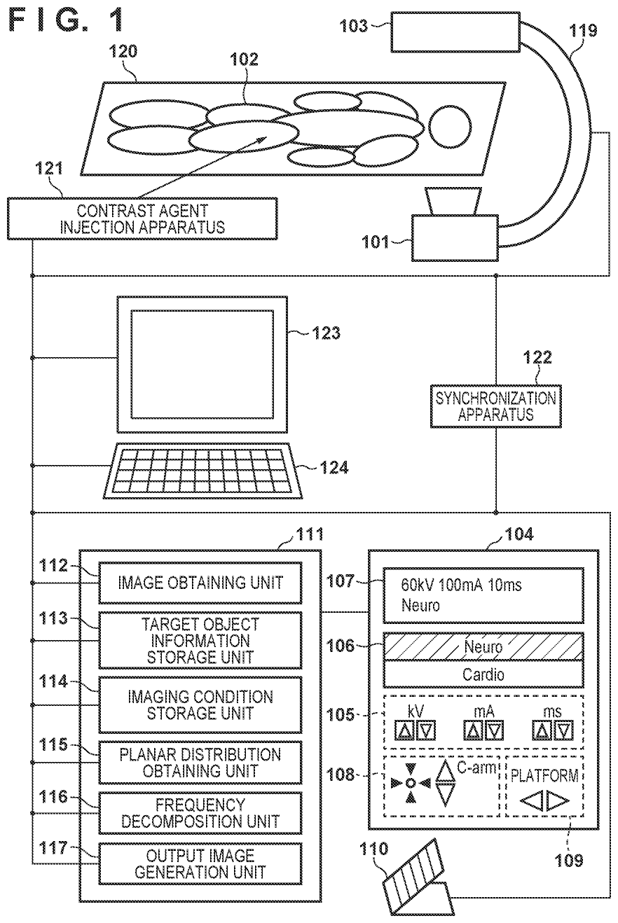

FIG. 1 shows a configuration of an X-ray imaging apparatus, which is an example of a radiation imaging apparatus according to the first embodiment. In FIG. 1, X-rays radiated from an X-ray tube 101 pass through an object 102 and are incident on a flat panel detector (hereinafter, “FPD 103”). The X-ray tube 101 may have an X-ray filter for removing low-energy X-rays. The FPD 103 includes a phosphor that converts X-rays into visible light, an image sensor that converts visible light into charge and voltage (there are a plurality of sensors, and one sensor corresponds to one pixel), and an image forming unit that further converts the converted voltage into a digital value image. The FPD 103 also includes an image correction unit that performs offset correction, gain correction, defect correction, or the like on the image obtained from the image forming unit. Note that the FPD 103 is configured to convert X-rays into visible light using the phosphor, but there is no limitation to this configuration. For example, an FPD that directly converts X-rays into voltage using cadmium telluride (CdTe) or amorphous selenium (a-Se) may alternatively be used. Alternatively, an FPD with a photon-counting detector that counts incident X-ray photons one by one may be used.

Conditions of X-rays to be radiated are determined by input by an operator from an operation panel 104. The operation panel 104 has a condition designation unit 105, an imaging area designation unit 106, a condition display unit 107, a C-arm control unit 108, and a platform control unit 109. The operator can use the condition designation unit 105 to designate the tube voltage, the tube current, and the radiation time. The operation panel 104 also has an adjuster (such as a dial) for adjusting image quality. The operator can set image quality that an image checker, such as a doctor, regards as most favorable, by operating the dial. The imaging area designation unit 106 holds imaging areas and X-ray conditions in association with each other in advance. The operator can call corresponding X-ray conditions by designating an imaging area.

The condition display unit 107 displays imaging conditions designated with the condition designation unit 105 and the imaging area designation unit 106, thus allowing the operator to confirm the set content. The operator can also use the C-arm control unit 108 and the platform control unit 109 to move vertically and horizontally or rotate a C-arm and a platform. An irradiation instruction unit 110 is a foot pedal. The operator can send an instruction to radiate X-rays to a computer 111 by stepping on the pedal.

An image output from the FPD 103 is transferred to the computer 111. The computer 111 serving as an image processing apparatus realizes functions of an image obtaining unit 112, a target object information storage unit 113, an imaging condition storage unit 114, a planar distribution obtaining unit 115, and a frequency decomposition unit 116. A display device 123 and an input device 124 are connected to the computer 111. The aforementioned image correction unit installed in the FPD 103 may alternatively be provided in the computer 111. Further, X-ray conditions may be stored in advance in the computer 111 instead of being input from the operation panel 104.

The image obtaining unit 112 takes the image generated by the FPD 103 into the computer 111. The target object information storage unit 113 obtains and stores information (object information) regarding the size (spatial frequency) of substances that are present inside an object and is to be imaged, such as a bone and a device like a stent. Content stored in the target object information storage unit 113 will be described later. The imaging condition storage unit 114 obtains information from the X-ray tube 101, the FPD 103, the operation panel 104, the input device 124, the C-arm 119, the platform 120, and a contrast agent injection apparatus 121 (these pieces of information are used as imaging conditions), and store the thus-obtained information.

Examples of the imaging conditions obtained by the imaging condition storage unit 114 include tube information, information regarding the FPD, imaging area information, object information, contrast agent information, C-arm information, and platform information. Tube information indicates the tube voltage, the tube current, the irradiation time, the size of a focal point, and the presence or absence of an X-ray filter, for example. Information regarding the FPD indicates X-ray accumulation time, for example. Imaging area information indicates an imaging area, such as the chest or head, for example. Object information indicates a patient's identification number, name, gender, age, height, weight, and the like that are input using the input device 124, for example. Contrast agent information indicates the type and concentration of a contrast agent, for example. C-arm information indicates the material, position, angle, and the like of the C-arm 119, for example. Platform information indicates the position and the like of the platform 120, for example.

The planar distribution obtaining unit 115 calculates a planar distribution related to a material based on an image obtained from the FPD 103 and prior information (e.g., X-ray energy spectrum during imaging). The planar distribution obtaining unit 115 obtains a decomposition image representing the planar distribution by means of energy subtraction, for example. An example of the planar distribution related to a material calculated by the planar distribution obtaining unit 115 is a spatial distribution of the thickness of each material. When an image obtained with absence of an object is I₀, and an image obtained with presence of an object is I, I/I₀ can be expressed by the following Expression 1.

$$\begin{matrix}[ Math 1 ] & \\ I / {I}_{0} = \frac{{\int }_{0}^{∞} N ( E ) exp \{ - {μ}_{1} ( E ) {d}_{1} - {μ}_{2} ( E ) {d}_{2} \} EdE}{{\int }_{0}^{∞} N ( E ) EdE} & ( Expression 1 )\end{matrix}$$

Here, E denotes energy, N(E) denotes an X-ray spectrum, μ₁(E) denotes a linear attenuation coefficient of a material 1, μ₂(E) denotes a linear attenuation coefficient of a material 2, d₁ denotes the thickness of the material 1, and d₂ denotes the thickness of the material 2. Of these, unknown variables are d₁ and d₂. Two independent equations can be generated by obtaining images with X-rays of two different levels of energy and substituting the obtained values into Expression 1. Thus, the values of the thicknesses d₁ and d₂ can be obtained by solving the two independent equations. Examples of the materials 1 and 2 may be a bone and a soft tissue. Processing for obtaining the spatial distribution of the thickness of each material is referred to as material decomposition.

Another example of a planar distribution related to a material is a spatial distribution of the effective atomic number and the area density. The spatial distribution of the effective atomic number and the area density can be expressed as follows.

$$\begin{matrix}[ Math 2 ] & \\ I / {I}_{0} = \frac{{\int }_{0}^{∞} N ( E ) exp \{ - μ ( {Z}_{eff} , E ) {D}_{eff} \} EdE}{{\int }_{0}^{∞} N ( E ) EdE} & ( Expression 2 )\end{matrix}$$

Here, E denotes the energy, N(E) denotes the X-ray spectrum, μ(Z_{eff}, E) denotes the mass attenuation factor at the effective atomic number Z_{eff} and energy E, and D_{eff} denotes the effective area density. Unknown variables in Expression 2 are the effective atomic number Z_{eff} and the effective area density Dar. Accordingly, two independent expressions can be generated by obtaining images with X-rays of two different levels of energy and substituting the obtained values for Expression 2, as in the case of obtaining the spatial distribution of thicknesses of each material. Thereafter, the values of the effective atomic number Z_{eff} and the effective area density D_{eff} can be obtained by solving the two independent equations. Processing for obtaining the effective atomic number and the effective area density is referred to as material identification.

The frequency decomposition unit 116 generates an image divided into a plurality of frequency components by performing frequency decomposition on the planar distribution related to the material obtained by the planar distribution obtaining unit 115. The thus-generated image of each frequency is hereinafter referred to as a band limitation image. The details of the frequency decomposition unit 116 will be described later. An output image generation unit 117 obtains a target object image in which a predetermined target object existing in the object 102 has been extracted, based on the band limitation image generated through frequency decomposition performed by the frequency decomposition unit 116.

The above functions of the image obtaining unit 112, the target object information storage unit 113, the imaging condition storage unit 114, the planar distribution obtaining unit 115, the frequency decomposition unit 116, and the output image generation unit 117 are implemented as software, and can be realized by one or more processors of the computer 111. Note that the computer 111 can be configured by general hardware including a central processing unit, a main storage device such as a DRAM, a secondary storage device such as a hard disk, a graphics processing unit for high-speed computing, a local area network (LAN) adapter, and so on. Note that some or all of the above functions may be realized by hardware, or may be realized by cooperation between hardware and software.

The input device 124 is, for example, a keyboard of a computer. The operator can use the input device 124 to input imaging area information and object information. In addition to input by the input device 124, imaging area information and object information may also be input from an external device connected to the LAN, for example, through a LAN adapter mounted in the computer 111. The target object information storage unit 113 obtains and stores imaging area information and object information.

The display device 123 is used to display output images. Use of a color display makes it possible to express, with colors, an image (hereinafter, “target object image”) of a target object generated based on the planar distribution related to the material and the band limitation image obtained after frequency decomposition is performed. Thus, diagnosability is expected to improve. Here, the target object is, for example, a small structure such as a guide wire, a stent, a microscopic blood vessel into which a contrast agent has been introduced, or lime attached to a vessel. In addition, the input device 124 and the display device 123 may include some of the functions of the operation panel 104. For example, input of conditions performed through the condition designation unit 105 or the imaging area designation unit 106 may alternatively be performed by the input device 124. Also, a display of imaging conditions such as the input tube voltage may alternatively be carried out by the display device 123 instead of the condition display unit 107.

The X-ray imaging apparatus may also include the contrast agent injection apparatus 121. The contrast agent injection apparatus 121 and the X-ray imaging apparatus can perform imaging in conjunction with each other by being connected to each other. The X-ray tube 101, the FPD 103, the operation panel 104, the irradiation instruction unit 110, and the computer 111 are connected to a synchronization apparatus 122. The synchronization apparatus 122 determines whether or not to carry out X-ray exposure based on the state of the FPD 103, whether or not the irradiation instruction unit 110 is being pressed, and the processing state of the computer 111.

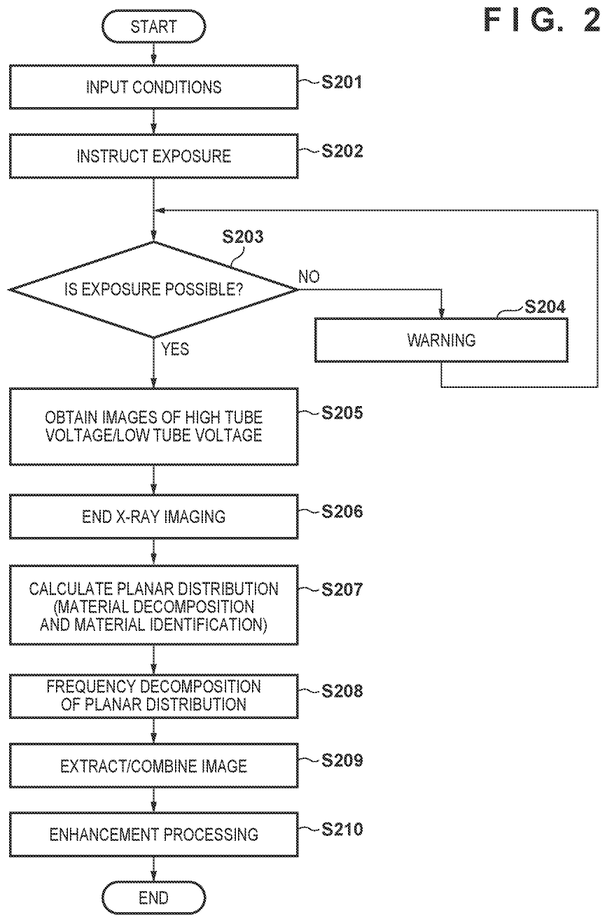

FIG. 2 is a flowchart showing X-ray imaging processing according to the present embodiment. First, the synchronization apparatus 122 and the imaging condition storage unit 114 accept input of imaging conditions (X-ray irradiation conditions etc.) by the operator (step S201). The imaging conditions are input through the condition designation unit 105 or the imaging area designation unit 106, for example. The imaging condition storage unit 114 stores the accepted imaging conditions in a memory. The imaging conditions stored by the imaging condition storage unit 114 are as mentioned above regarding FIG. 1.

Next, the synchronization apparatus 122 accepts an X-ray irradiation instruction from the irradiation instruction unit 110 given by the operator (step S202), and then determines whether or not exposure is possible (step S203). The synchronization apparatus 122 determines whether or not to carry out exposure based on, for example, the states of the X-ray tube 101, the FPD 103, the operation panel 104, the irradiation instruction unit 110, the computer 111, and the contrast agent injection apparatus 121. If it is determined that exposure cannot to be carried out (NO in step S203), the synchronization apparatus 122 displays a warning in the display device 123 (step S204). Note that the synchronization apparatus 122 may alternatively notify the computer 111 of the result of determining that exposure cannot be carried out, and the computer 111 may display a warning in the display device 123.

On the other hand, if the synchronization apparatus 122 determines that exposure can be carried out (YES in step S203), the synchronization apparatus 122 sends an X-ray irradiation instruction signal to the X-ray tube 101 and the FPD 103. Thus, the X-ray tube 101 starts radiating X-rays, the FPD 103 starts X-ray imaging, and the image obtaining unit 112 obtains an X-ray image as a radiation image (step S205). Here, the image obtaining unit 112 obtains two or more radiation images (X-ray images) obtained by imaging the object using of radiation (X-rays) of different levels of energy. In the present embodiment, an X-ray image (high tube voltage image) obtained by radiating X-rays with a first tube voltage and an X-ray image (low tube voltage image) obtained by radiating X-rays with a tube voltage that is lower than the first tube voltage are obtained (step S205). After a necessary number of images has been obtained, X-ray imaging ends (step S206).

Next, the planar distribution obtaining unit 115 generates a decomposition image representing the planar distribution related to materials through material decomposition or material identification from two or more X-ray images obtained by imaging an object that contains the target object by means of radiation of different levels of energy. In the present embodiment, the planar distribution obtaining unit 115 calculates and generates a decomposition image representing the planar distribution related to the materials from the high tube voltage image and the low tube voltage image (step S207). Next, the frequency decomposition unit 116 performs frequency decomposition on the planar distribution (decomposition image) related to the material calculated in step S207 (step S208). For example, the planar distribution obtaining unit 115 obtains two decomposition images, which are a bone image and a soft tissue image, as the planar distributions related to the materials. The bone image contains a bone and a target object (a contrasted blood vessel etc.). The frequency decomposition unit 116 performs frequency decomposition on the bone image and thus decomposes the bone image into an image of the target object (a band limitation image in which the target object is enhanced) and a bone image (a band limitation image in which the bone is enhanced).

Next, the output image generation unit 117 extracts an image (target object image) that contains the target object from the band limitation image obtained by decomposition by the frequency decomposition unit 116, or performs combination to obtain the target object image (step S209). The present embodiment describes an example of extraction (the second and subsequent embodiments will describe examples of combination later). The output image generation unit 117 then performs enhancement processing on the extracted or combined target object image (step S210). Examples of enhancement processing include processing for displaying the target object in color, processing for enhancing the contrast, and the like.

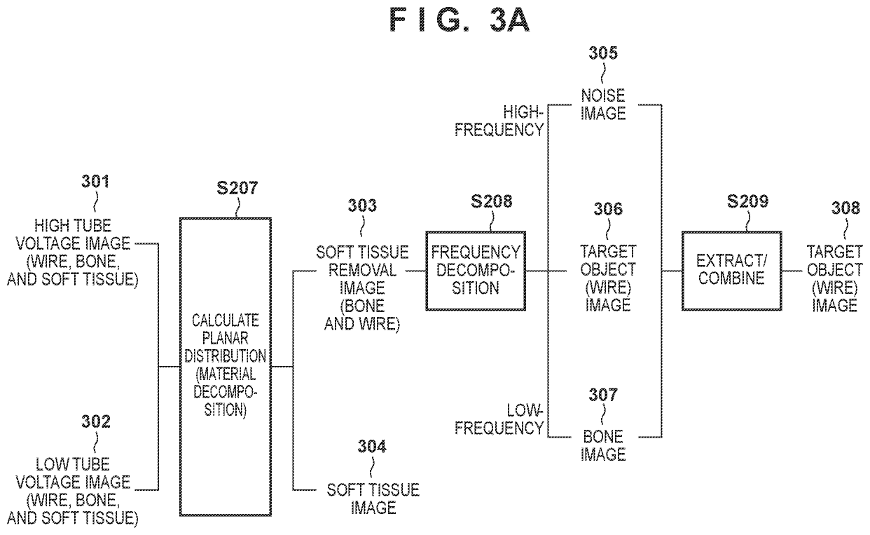

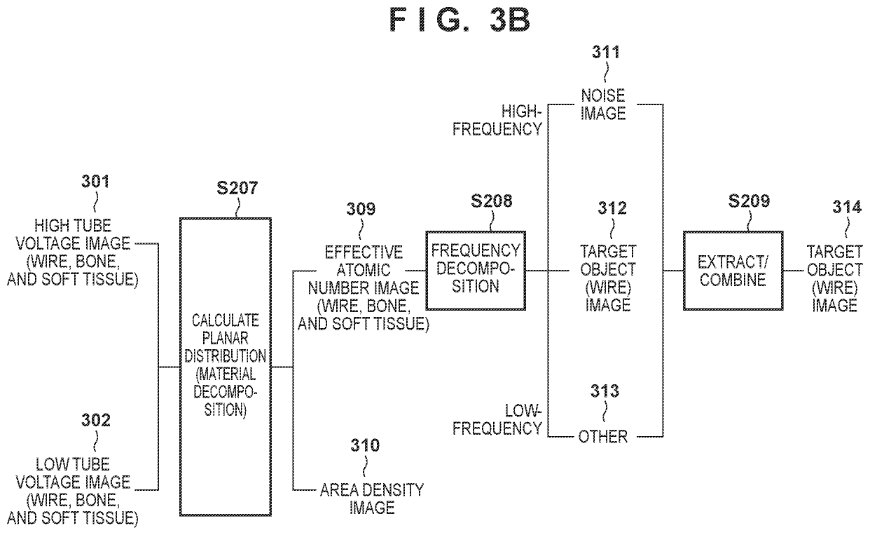

FIGS. 3A and 3B illustrate processing in steps S207 to S209 in FIG. 2 in more detail. It is assumed that in FIGS. 3A and 3B, the image contains three types of materials, namely a guide wire (which may alternatively be a stent, a coil etc.) serving as a target object, a bone, and a soft tissue. IVR (Interventional Radiology) procedures often involve these three materials. The first embodiment describes a configuration for providing an image in which these three materials are separated. Note that four materials, namely these three materials plus a contrast agent may be contained. The third and fourth embodiment will later describe configurations for providing an image in which four materials are separated.

FIG. 3A illustrates a process of three-materials decomposition through material decomposition and frequency decomposition. First, the planar distribution obtaining unit 115 generates a decomposition image through material decomposition (step S207). For example, the planar distribution obtaining unit 115 generates two decomposition images, which are a soft tissue removal image 303, which serves as a bone image, and a soft tissue image 304, based on the high tube voltage image 301 and the low tube voltage image 302. By setting the line attenuation coefficient of the bone as the line attenuation coefficient μ₁ (E) of the material 1 and setting the line attenuation coefficient of the soft tissue as the line attenuation coefficient μ₂ (E) of the material 2 in the above-described Expression 1, the soft tissue removal image 303 is obtained as an image of the material 1, and the soft tissue image 304 is obtained as an image of the material 2. The soft tissue removal image 303 contains the bone and the wire, and the soft tissue image includes the soft tissue.

Next, the frequency decomposition unit 116 performs frequency decomposition on the soft tissue removal image 303 to divide the soft tissue removal image 303 into a plurality of frequency components, and generates a plurality of band limitation images (step S208). According to an example, here, the highest-frequency band limitation image is an image that mainly contains noise components (a noise image 305), and the next highest-frequency band limitation image is an image that most strongly contains guide wire components (a wire image 306). The band limitation image of low frequency components is an image that strongly contains the bone components (a bone image 307). The output image generation unit 117 extracts (selects) a band limitation image (wire image 306) that most strongly contains the guide wire components, and generates a target object image 308 based on the extracted band limitation image (step S209). The band limitation image is extracted based on information stored in the target object information storage unit 113 and the imaging condition storage unit 114.

As described above, in the case of performing three material-decomposition through material decomposition and frequency decomposition, the three-material decomposition can be performed even though two types of images, namely the high tube voltage image and the low tube voltage image are input. In other words, a greater number of types of materials than the number of input X-ray images can be discriminated by performing frequency decomposition on a decomposition image. Note that not all decomposition images of the three materials are necessarily used. Examples of the use of the images of three materials include highlighting the wire using only the wire image out of the extracted images, and displaying an image in which only the bone is removed using the soft tissue image and the wire image.

FIG. 3B illustrates the process of extracting the wire through material identification and frequency decomposition. In step S207 in FIG. 3B, the planar distribution obtaining unit 115 generates a decomposition image through material identification. For example, the planar distribution obtaining unit 115 generates two decomposition images, which are an effective atomic number image 309 and an area density image 310, based on the high tube voltage image 301 and the low tube voltage image 302. The effective atomic numbers of the bone, the wire, and the soft tissue appear in the effective atomic number image 309. The effective area density of each material appears in the area density image 310.

The frequency decomposition unit 116 performs frequency decomposition on the effective atomic number image 309, and generates a plurality of band limitation images having respective frequency components (step S208). The highest-frequency image strongly contains noise components (a noise image 311), and the next highest-frequency image strongly contains the guide wire components (a wire image 312), as in the case of the material decomposition described above with reference to FIG. 3A. The low-frequency image strongly contains components other than the guide wire (an image 313 of other components). The output image generation unit 117 extracts (selects) the wire image 312 in which the target object (guide wire) appears based on the information stored in the target object information storage unit 113 and the imaging condition storage unit 114, and generates a target object image 314 based on the extracted wire image 312 (step S209).

As described above, when the target object is extracted through material identification and frequency decomposition, three types of images, namely the effective atomic number image, the area density image, and the extracted target object image are generated even though two types of images, namely the high tube voltage image and the low tube voltage image are input. In other words, a greater number of images than the number of input X-ray images are generated by applying frequency decomposition to the decomposition image. In the case of using material identification and frequency decomposition, the effective atomic number image, the area density image, and one or more target object images are generated. Note that not all the decomposition images are necessarily used, as in the case of the material decomposition.

When the target object image is generated by performing frequency decomposition on the soft tissue removal image or the effective atomic number image, the target object, such as a wire, appears with higher pixel values than the surroundings of the target object image (the wire has a positive thickness/high effective atomic number). Accordingly, pixels corresponding to the wire are extracted by extracting pixels with pixel values not smaller than a certain threshold in the target object image 308 or 314 in the enhancement processing (step S210), for example, and the wire can be enhanced using the extraction results.

In step S209, the output image generation unit 117 extracts (selects) a band limitation image based on information stored in the target object information storage unit 113 and the imaging condition storage unit 114. For example, it is assumed that the guide wire needs to be enhanced in the current situation. Note that the determination of such a situation can be performed based on information stored in the imaging condition storage unit 114 that is input from the imaging area designation unit 106 or the like. In this case, the frequency to be enhanced is determined as follows, for example.

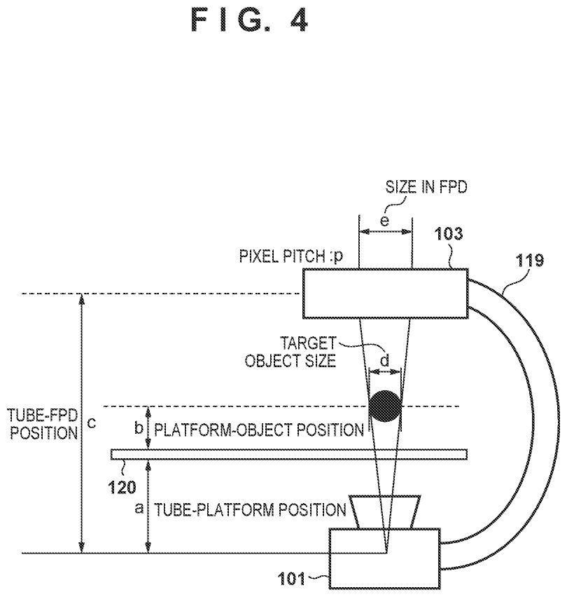

It is assumed that the tube-platform position is a [mm], the platform-object position is b [mm], the tube-FPD position is c [mm], and the size of the target object (guide wire) is d [mm], as shown in FIG. 4. It is also assumed that the size of the guide wire on the FPD 103 is e [mm], and the pixel pitch of the detector of the FPD 103 is p [mm]. Note that the pixel pitch p may vary depending on the imaging mode, such as binning. In this case, the pixel-equivalent thickness (x [pixels]) of the guide wire appearing in the image is expressed by Expression 3 below.

$$\begin{matrix}[ Math 3 ] & \\ x = \frac{e}{p} = \frac{1}{p} \times \frac{c}{a + b} d & ( Expression 3 )\end{matrix}$$

Therefore, the frequency to be enhanced may be around 1/x [1/pixels], and an image close to this frequency band is selected. More simply, the frequency to be enhanced may be determined in advance for each imaging mode, and the frequency corresponding to the imaging mode may be loaded at the beginning of imaging. If the frequency to be enhanced is calculated in advance, information regarding this calculation is stored in the target object information storage unit 113.

Note that the tube-platform position is calculated based on position information regarding the C-arm 119 and the platform 120 that is stored in the imaging condition storage unit 114. The platform-object position can be estimated from the position information regarding the platform 120 and body shape information regarding the object 102. Body shape information regarding the object 102 is stored in the target object information storage unit 113. Alternatively, a default value (which is stored in the imaging condition storage unit 114) may be used as the platform-object position. The tube-FPD position is calculated based on the position information regarding the C-arm 119 that is stored in the imaging condition storage unit 114. The size of the target object (guide wire) is stored in the target object information storage unit 113, and a typical value is about 0.8 [mm]. The pixel pitch is determined by the specifications of the FPD 103 and the imaging mode, such as binning. The specifications of the FPD 103 and the imaging mode are stored in the imaging condition storage unit 114. The pixel pitch is 0.2 mm, for example.

Here, reasons why the target object such as a guide wire can be extracted by performing frequency decomposition on the soft tissue removal image or the effective atomic number image is described. In most cases, a target object that is a substance introduced into the human body, such as a guide wire or a stent, has a physical size that is significantly smaller than a sternum or the like, for example. Further, many of these target objects are made of metal, such as stainless steel (iron). For this reason, the target object is generated very finely with strong contrast, compared to a bone image, in the soft tissue removal image. In the effective atomic number image as well, a target object, such as a guide wire or a stent, appears very finely with strong contrast compared to a bone and a soft tissue due to its large atomic number. Since an image with a fine structure and strong contrast strongly appears as high-frequency components, target particles of a guide wire, a stent, or the like can be extracted from an image in which a plurality of materials are mixed by extracting the high-frequency components.

In contrast, when frequency decomposition is performed on an accumulated image without performing material decomposition or material identification in advance, it is difficult to extract only the target object, such as a catheter or a stent, because a pulmonary blood vessel or the like of a soft tissue appears as the high-frequency components. Accordingly, it is important to use both the planar distribution obtaining unit 115 and the frequency decomposition unit 116 in order to extract a target object such as a catheter or a stent.

In the case of a fine substance with a large effective atomic number, the material thereof can be separated by the frequency decomposition unit 116 performing frequency decomposition on the planar distribution obtained by the planar distribution obtaining unit 115. Examples of the extracted substances include artificial devices that is inserted into the human body from outside, such as a puncture needle, a guide wire, a stent, a stent graft, a catheter, a coil placed in the brain, an electronic device (medical or non-medical), a prosthetic limb or denture, and a bullet. A marker or the like that is present in these devices can also be separated. A contrast agent (a blood vessel containing a contrast agent), lime adhering a blood vessel, and the like can also be separated similarly. Lime has an effective atomic number similar to that of bone, but has a structure finer than bone. Therefore, lime can be extracted with this method.

Note that PTL 2 describes an example of using an unsharp mask in an angiography apparatus that uses two levels of energy. However, using an unsharp mask to enhance a fine structure enhances not only edges of an image to be observed (e.g., lime) but also edges of a bone. It is therefore difficult to extract only the target object. In contrast, in the present embodiment, an image of the target object can be extracted by extracting a specific spatial frequency band of the image based on the spatial frequency possessed by the target object, i.e., the size of the target object. In other words, in the present embodiment, an image in which the target object is estimated to appear is extracted based on thickness and size information (frequency information) regarding a material that can be predicted to some extent in advance (step S209). Thus, a target object such as a stent, a guide wires, or lime can be effectively extracted.

In the previously described image containing three materials, namely a guide wire, a bone, and a soft tissue, the guide wire appears with a negative thickness in the soft tissue image in some cases. This is because the thickness of the soft tissue becomes thin in a portion where the guide wire is located. In that case, the guide wire can be extracted by performing frequency decomposition on the soft tissue image 304 (step S208). For example, a pulmonary vessel appears with a positive thickness, while a guide wire appears with a negative thickness. Thus, the pulmonary vessel, which is a fine structure, can be separated from the guide wire. Furthermore, the guide wire can be extracted more clearly by additionally performing threshold processing in which a soft tissue with a thickness below a certain level is judged to be the guide wire. As for the area density image as well, a portion where the wire is present shows a characteristic peak, and the wire can therefore be extracted by setting a threshold.

In FIGS. 3A and 3B, an image in which the target object appears is selected from three band limitation images. In general, however, one or more images are selected from two or more band limitation images. Note that the third embodiment will describe an image of obtaining two target object images by decomposition into four materials.

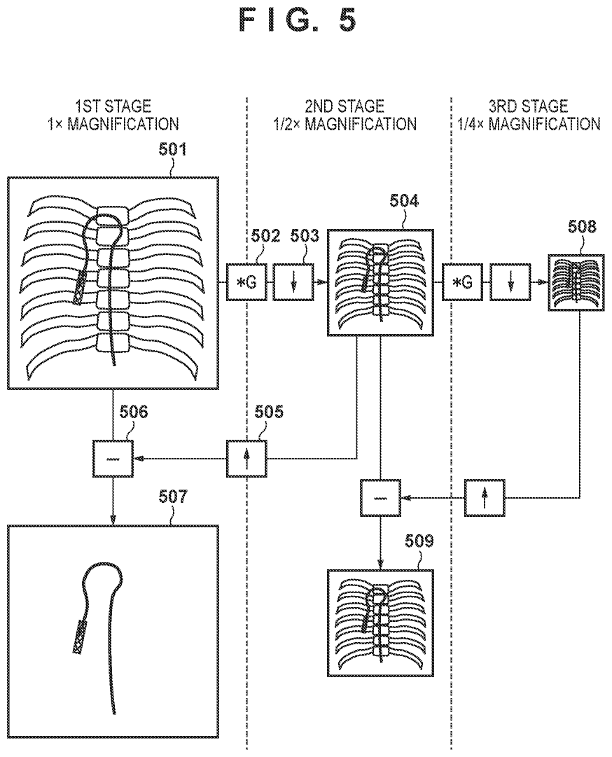

FIG. 5 shows an example of frequency decomposition processing performed by the frequency decomposition unit 116. FIG. 5 shows an example of performing frequency decomposition using a so-called Laplacian pyramid method. An image 501 is a decomposition image representing a planar distribution related to the materials obtained from the planar distribution obtaining unit 115. Examples of decomposition images include a bone image obtained after material decomposition is performed, or an effective atomic number image obtained after material identification is performed. The image 501 is subjected to a Gaussian filter 502 and then reduced through reduction processing 503. Then, an image 504 is obtained. The image 504 is enlarged through enlargement processing 505, and a difference between the original image 501 and the enlarged image 504 is calculated through subtraction processing 506. Then, an image 507, which is a band limitation image, is obtained. Similarly, an image 508 is obtained by applying the Gaussian filter and reduction processing to the image 504, and a difference between the reduced image 508 and the image 504 is calculated through subtraction processing. Thus, an image 509, which is a band limitation image, is obtained. Thus, band limitation images in various frequency bands can be obtained by repeating the reduction, enlargement, and subtraction processes. A band limitation image in a specific frequency band corresponding to the target object is obtained out of a plurality of thus-obtained band limitation images. If one of the images 509 and 508, which have been reduced, is selected, the selected image is enlarged to the original size.

As described above, according to the first embodiment, a decomposition image can further be decomposed into images of a plurality of target objects by performing frequency decomposition on the decomposition image. In other words, images corresponding to a greater number of materials than the number of input radiation images can be obtained. Thus, many types of materials can be extracted without increasing the number of times of imaging.

Note that the frequency decomposition method is not limited to that described in the above example. For example, frequency decomposition can be performed by using Fourier transform or by obtaining a derivative of an image (a difference from neighboring pixels). In the above embodiment, a plurality of band limitation images corresponding to a plurality of frequencies are generated, and a band limitation image with a frequency corresponding to a target object is selected. However, there is no limitation to this configuration. If the frequency decomposition unit 116 can generate a band limitation image corresponding to a desired frequency, only the band limitation image corresponding to the target object may be generated by the frequency decomposition unit 116 and used as an output image.

There are cases where the target object to be observed by the operator changes during the procedure. For example, if a blood vessel is observed using a contrast agent and then stent placement starts, the target object to be observed by the operator changes from the contrast agent to the stent. Accordingly, the band limitation image extracted by the output image generation unit 117 may also change during the procedure. A signal input by an operator through the input device 124, for example, can be used as a trigger for notifying the timing of such a change. For example, the frequency for selecting a band limitation image is changed in accordance with a change in the target object to be observed.

### Second Embodiment

In the first embodiment, one image is extracted as a target object image from generated band limitation images in a plurality of frequency bands. In the second embodiment, a target object image is generated by combining a plurality of band limitation images while weighting these images. This method, in which images in a plurality of frequency bands are used, enables a target object to be stably extracted even when the size of the target object to be checked changes.

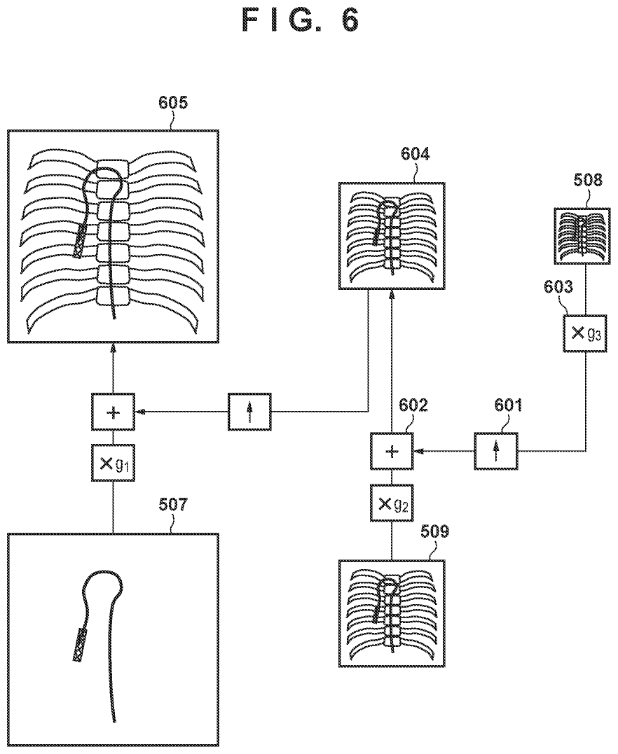

The apparatus configuration and the processing flow are the same as those of the first embodiment, except for step S209 in FIG. 2. In the second embodiment, a plurality of band limitation images are combined instead of extracting one of a plurality of band limitation images in step S209. FIG. 6 is a flowchart illustrating combination processing in step S209. Images 507, 508, and 509 are band limitation images obtained by performing frequency decomposition described with reference to FIG. 5.

The output image generation unit 117 multiplies the image 508 by a weighting coefficient g₃ through weighting processing 603, and enlarges the obtained weighted image through enlargement processing 601 to obtain an enlarged image. Also, the output image generation unit 117 multiplies the image 509 by a weighting coefficient g₂ through weighting processing to obtain a weighted image. The output image generation unit 117 performs addition processing 602 to add the image enlarged through enlargement processing 601 to the weighted image 509 to obtain a combined image 604. As a result, the mixing ratio between the image 509 and the image 508 can be changed in the combination to obtain the combined image 604, and a specific frequency band can then be enhanced. Similarly, the output image generation unit 117 performs addition processing to add a weighted image obtained by multiplying the image 508 by a weighting coefficient g₁ through weighting processing to an enlarged image obtained by enlarging the combined image 604, and thus obtains a combined image 605. The mixing ratio between the image 509 and the combined image 604 can also be changed in the combination to obtain the combined image 605, and a specific frequency band can thus be enhanced.

In the case of particularly enhancing a stent in the situation in FIG. 6, for example, the coefficient g₁ related to the image 507, in which the stent appears most clearly, is increased, among the weighting coefficients g₁, g₂, and g₃. Note that weighting coefficients corresponding to target objects to be observed are stored in the target object information storage unit 113.

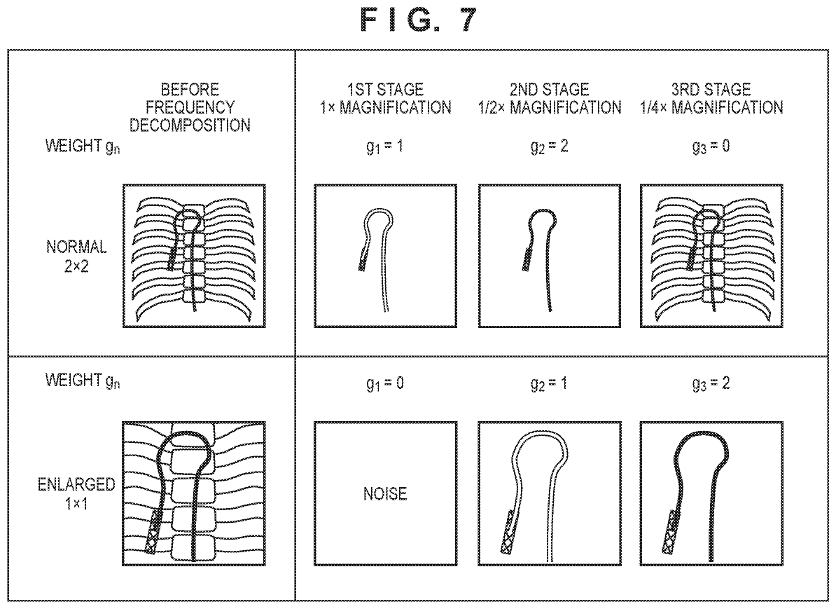

In the second embodiment, the ratio of a weighting coefficient gᵢ (i: integer) may be varied in accordance with the imaging conditions. FIG. 7 shows examples of weighting coefficients. Here, examples in the cases of normal imaging (2×2 binning) and enlargement imaging (1×1 binning) are shown. In the case of 1×1 binning, the pixel pitch is small. Therefore, the pixel equivalent thickness [pixel] of the guide wire appearing in an image is large (see Expression 3). For this reason, the frequency band [1/pixels] in which the wire begins to appear is different from that in the case of normal 2×2 binning. Accordingly, the weighting coefficient gᵢ (i: integer) also needs to be changed depending on the state of binning. In addition, the weighting coefficient gᵢ also needs to be changed in accordance with the enlargement ratio (which is determined by the positions or the like of the tube, the platform, the object, and the detector). This weighting coefficient may be calculated based on information (examples of which is described in the first embodiment) stored in the target object information storage unit 113 and the imaging condition storage unit 114, or may be stored in advance in the form of a table.

The imaging conditions include imaging area information (selection of an area to be imaged), as described in the first embodiment. The weighting coefficient gᵢ may also be set based on the imaging area information. When, for example, cerebral blood vessels are imaged, higher-resolution images tend to be required than in imaging of other areas. Accordingly, the weight of the band limitation image on the high-frequency side is increased when frequency decomposition is performed. Also, the dose obtained based on tube information or the like may also be used as a parameter for setting the weighting coefficient gᵢ. For example, the larger the dose is, the more easily a fine structure can be observed. Therefore, the coefficient gᵢ may be set such that a higher-frequency image is weighted.

Note that the target object to be observed by the operator may also change in the second embodiment. Thus, images to be combined may also be changed. For this reason, there are cases where the weighting coefficient is changed during the procedure. An example of a trigger for notifying the timing of a change is a signal input by the operator through the input device 124.

### Third Embodiment

The first and second embodiments have described a configuration for decomposition into three materials (e.g., a soft tissue, a bone, and a stent). The third embodiment will describe an example of a configuration for decomposition into four materials (e.g., a soft tissue, a bone, a stent, and a contrast agent). The third embodiment is a variation of the second embodiment (combination of a plurality of band limitation images).

In general, a stent is constituted by thin metal wires and is arranged in a blood vessel. Meanwhile, a contrast agent, after being injected into the human body, spreads throughout a blood vessel and appears as a contrast vessel. Comparing the metal thickness of the stent to that of the contrast vessel, the metal of the stent is significantly thinner than the contrast vessel and appears in higher-frequency images. Therefore, the stent can be extracted by giving a higher weight to a high-frequency image out of the band limitation images obtained by performing frequency decomposition, and the contrast vessel (contrast agent) can be extracted by giving a higher weight to an image with a frequency lower than that of the stent. In the third embodiment, it is assumed below that an object 102 that contains four materials, namely a stent, a contrast agent, a bone, and a soft tissue, is imaged.

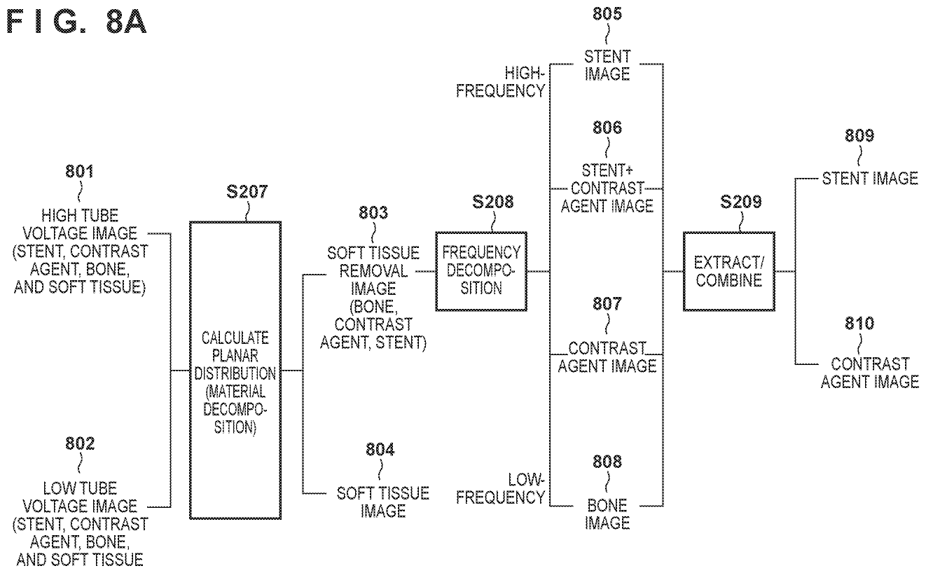

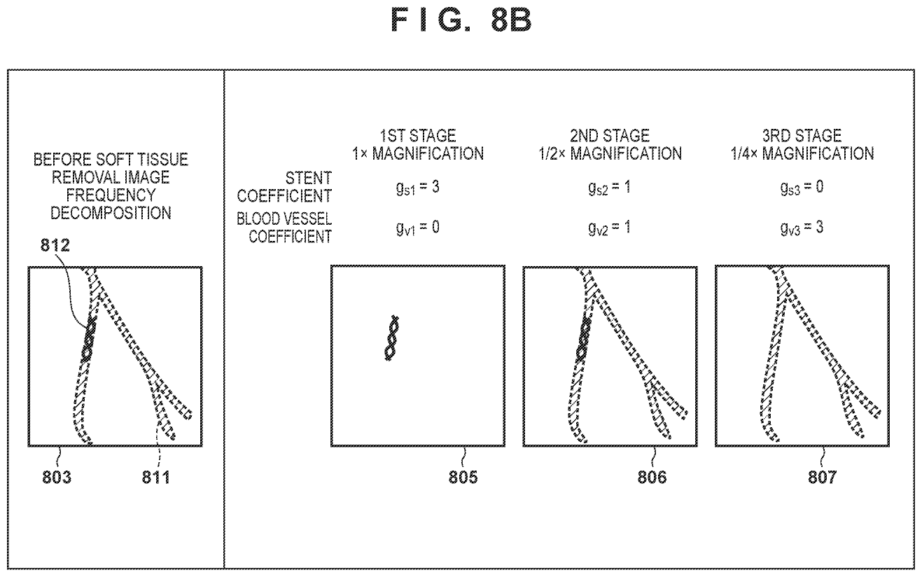

FIG. 8A illustrates a process of extracting a stent and a contrast vessel according to the third embodiment. First, a high tube voltage image 801 and a low tube voltage image 802 are shot. A difference from the high tube voltage image 301 and the low tube voltage image 302 described in the first embodiment lies in that the number of materials that need to be discriminated is increased by one to a total of four. The planar distribution obtaining unit 115 performs material decomposition using the high tube voltage image 801 and the low tube voltage image 802 (step S207), and generates a soft tissue removal image 803 and a soft tissue image 804. Although an example of using material decomposition is described here, there is no limitation thereto. For example, the effective atomic number and the area density image may alternatively be generated using material identification such as that shown in FIG. 3B.

The frequency decomposition unit 116 performs frequency decomposition on the soft tissue removal image 803 and generates band limitation images (step S208). The third embodiment describes an example of decomposition into four band limitation images. Specifically, the soft tissue removal image 803 is decomposed into four band limitation images, which are an image 805 in which a stent appears, an image 806 in which the stent and a contrast agent (contrast vessel) appear together, an image 807 in which the contrast agent appears, and a bone image 808, in descending order of frequency. The output image generation unit 117 obtains two target object images, namely a stent image 809 and a contrast agent image 810, by performing selection processing or combination processing (step S209).

FIG. 8B illustrates combination processing performed by the output image generation unit 117. In the soft tissue removal image 803, 811 denotes a blood vessel into which the contrast agent has been introduced, and 812 denotes the stent placed onto the blood vessel. The soft tissue removal image 803 is generated by the planar distribution obtaining unit 115, as described with reference to FIG. 8A. For example, an effective atomic number image may be used instead of the soft tissue removal image 803, as mentioned above.

The frequency decomposition unit 116 performs frequency decomposition on the soft tissue removal image 803 (step S208), as described with reference to FIG. 8A. As a result, four band limitation images, namely the image 805 in which the stent appears, the image 806 in which the stent and the contrast agent (contrast vessel) appear together, the image 807 in which the contrast agent appears, and the bone image 808, are generated (the bone image 808 is not shown in FIG. 8B). To extract the stent and the contrast vessel from the images 805, 806, and 807, the target object information storage unit 113 holds a plurality of sets of weighting coefficients corresponding to different target objects (e.g., the stent and the contrast vessel). Specifically, a set gₛᵢ of the weighting coefficient (stent coefficient) for extracting the stent and a set gᵥᵢ (i is an integer) of the weighting coefficient (blood vessel coefficient) for extracting the contrast vessel are held. The stent appears in a high-frequency image, and the contrast vessel appears in an image with a frequency lower than that of the stent image. Thus, the stent coefficient is given a higher weight on the high frequency, and the blood vessel coefficient is given a higher weight on a frequency lower than that of the stent coefficient. The stent image 809 and the contrast agent image 810 are generated by performing combination processing, such as that described with reference to FIG. 7, using the respective sets of coefficients.

In general, the contrast agent is introduced into the human body in the middle of the procedure. Therefore, four-material decomposition described in the third embodiment may be used in the middle of the procedure. In other words, three-material decomposition described in the first or second embodiment may be performed at the initial stage in a sequence of procedure, and may be switched to four-material decomposition described in the third embodiment upon the contrast agent being introduced. That is to say, the number of materials to be separated may be switched during the procedure. For example, the frequency decomposition unit 116 generates band limitation images the number of which corresponds to the number of types of target objects to be extracted, and switches the number of band limitation images to be generated in response to a predetermined trigger. An example of a trigger for this switching is a signal input by the operator through the input device 124. Another example is a signal given from the contrast agent injection apparatus 121. Yet another example is a signal issued by a contrast agent detection unit (which detects the presence or absence of a contrast agent in an image) that is additionally provided to the computer 111. In this case, the contrast agent detection unit can detect the presence or absence of a contrast agent by detecting a significant difference in contrast from the previous frame, for example.

Although the third embodiment has described separation between a stent and a contrast agent, this embodiment is also applicable to combinations of other target objects; e.g., a combination of a contrast agent and a catheter, a combination of a stent and a guide wire, and a combination of a guide wire and lime. Furthermore, decomposition into five or more give target objects is enabled by preparing three or more sets of weighting coefficients for decomposition and performing three or more combination processes.

### Fourth Embodiment

The third embodiment has described a configuration for decomposition into a soft tissue, a stent, a contrast agent, and a bone by performing frequency decomposition. However, if a contrast vessel is thick, there are cases where the frequency band in which the contrast vessel appears is close to the frequency band in which the bone appears. In that case, there is a possibility that decomposition into the bone and the contrast agent cannot be sufficiently performed by means of material decomposition with frequency decomposition only. The fourth embodiment will describe a configuration for improving the accuracy of four-material decomposition by introducing mask processing to remove the bone.

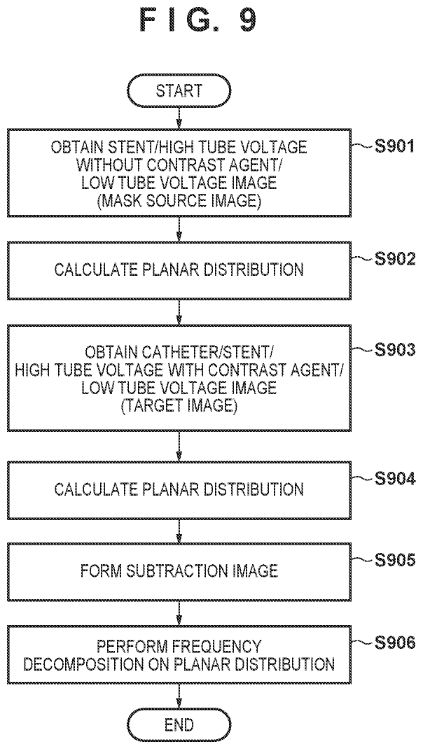

FIG. 9 is a flowchart illustrating imaging processing according to the fourth embodiment. Note that processing (steps S901 to S906) shown in FIG. 9 replaces processing in steps S205 to S208 in FIG. 2. In the fourth embodiment, decomposition into a stent, a contrast agent, a bone, and a soft tissue is performed. In steps S901 and S902, a decomposition image serving as a mask image is generated from two or more radiation images shot by imaging the object 102 in a state of not containing a target object with radiation of different levels of energy. Specifically, first, the image obtaining unit 112 obtains a high tube voltage image and a low tube voltage image (which is called a mask source image) that are shot before the stent and the contrast agent are introduced (step S901). Subsequently, the planar distribution obtaining unit 115 derives a planar distribution related to the materials and generates two decomposition images, namely a soft tissue image and a soft tissue removal image (step S902). Since images shot before the stent and the contrast agent are introduced are used, the soft tissue removal image obtained here is a bone image without any target object. The thus-obtained soft tissue removal image is used as a mask image.

Subsequently, the image obtaining unit 112 obtains a high tube voltage image and a low tube voltage image that are shot at the timing of placing the stent and introducing the contrast agent (step S903). The images obtained in this step contains four materials, namely the soft tissue, the bone, the contrast agent, and the stent. Subsequently, the planar distribution obtaining unit 115 derives a planar distribution related to the materials, and generates two images, namely a soft tissue image and a soft tissue removal image (step S904). Not only the bone but also the contrast agent and the stent appear in the soft tissue removal image obtained here.

A soft tissue removal image serving as a bone mask image (steps S901 and S902) and a soft tissue removal image that contains the bone and the target objects (contrast agent and stent) are obtained (steps S903 and S904) up to the above. Here, when attention is paid to movement of the bone, the contrast agent, and the stent, the contrast agent and the stent greatly move in conjunction with the beating of the heart, for example, while the bone hardly moves. For this reason, an image that only contains the contrast agent and the stent can be extracted by subtracting the soft tissue removal image (mask image) that only contains the bone obtained in step S902 from the soft tissue removal image that contains three materials, namely the bone, the contrast agent, and the stent, that is obtained in step S904. The frequency decomposition unit 116 removes bone components from the soft tissue removal image obtained in step S904 by performing this subtraction, and obtains a subtraction image in which only the contrast agent and the stent remain (step S905).

Further, the thin wires that constitute the stent are significantly different in size from the blood vessel in which the contrast agent flows, as described in the third embodiment. Accordingly, decomposition into the stent and the contrast agent is realized by applying frequency decomposition described in the third embodiment. The frequency decomposition unit 116 performs such frequency decomposition, and generates a plurality of band limitation images that include band limitation images corresponding to the stent image and the contrast agent image (step S906). Thereafter, the output image generation unit 117 extracts the stent image and the contrast agent image from the plurality of band limitation images.

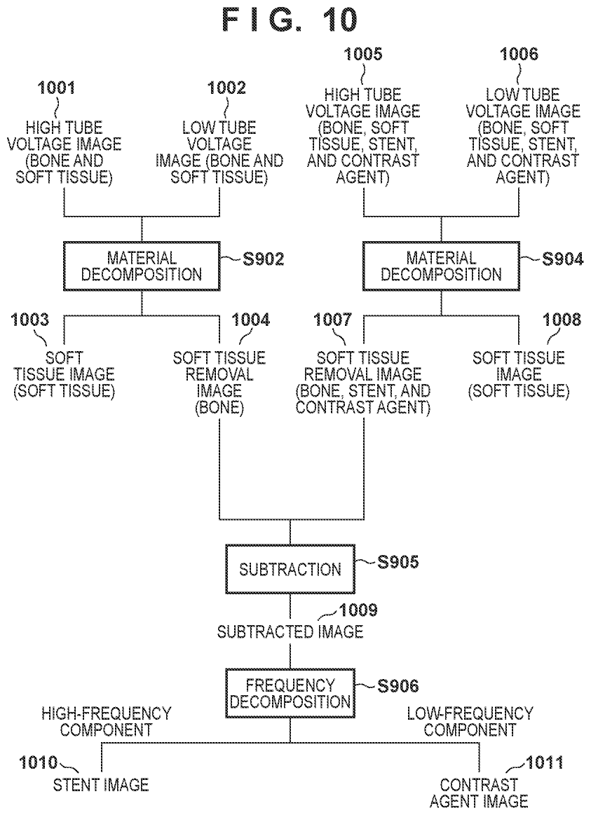

FIG. 10 is a diagram for illustrating processing in steps S902 to S906 described in FIG. 9 in more detail. The image obtaining unit 112 obtains a high tube voltage image 1001 and a low tube voltage image 1002 that contain only the bone and the soft tissue (and does not contain the stent or the contrast agent) (step S901). The planar distribution obtaining unit 115 derives a planar distribution related to the materials (material decomposition in this example) for the high tube voltage image 1001 and the low tube voltage image 1002 (step S902). Thus, a soft tissue image 1003 corresponding to the soft tissue image and a soft tissue removal image 1004 corresponding to the bone are generated.

Next, the image obtaining unit 112 obtains a high tube voltage image 1005 and a low tube voltage image 1006 that contain the bone, the soft tissue, the stent, and the contrast agent (step S903). The planar distribution obtaining unit 115 derives a planar distribution related to the materials (material decomposition in this example) for the high tube voltage image 1005 and the low tube voltage image 1006 (step S904). Thus, a soft tissue image 1008 corresponding to the soft tissue image and a soft tissue removal image 1007 corresponding to the bone and the target objects (the contrast agent and the stent) are generated.

Of the bone, the contrast agent, and the stent, the bone moves only slightly and therefore appears at the same position in the soft tissue removal image 1004 and the soft tissue removal image 1007. Therefore, the planar distribution obtaining unit 115 obtains a subtraction image 1009 from which the bone has been removed, by subtracting the soft tissue removal image 1004 from the soft tissue removal image 1007 (step S905). Further, the frequency decomposition unit 116 separates the stent from the contrast agent by performing frequency decomposition using the difference in spatial frequency characteristics between the stent and the contrast agent (step S906). Thus, a stent image 1010 and a contrast agent image 1011 are obtained.

In the above description, the frequency decomposition unit 116 first performs subtraction (step S905) and then performs frequency decomposition. However, the frequency decomposition unit 116 may alternatively perform frequency decomposition first and then perform subtraction. In this case, the frequency decomposition unit 116 first performs frequency decomposition on the soft tissue removal image 1007 and generates a stent image and a bone-contrast agent image. Thereafter, the frequency decomposition unit 116 generates a contrast agent image 1011 by subtracting the soft tissue removal image 1004 serving as a bone mask image from the bone-contrast agent image.

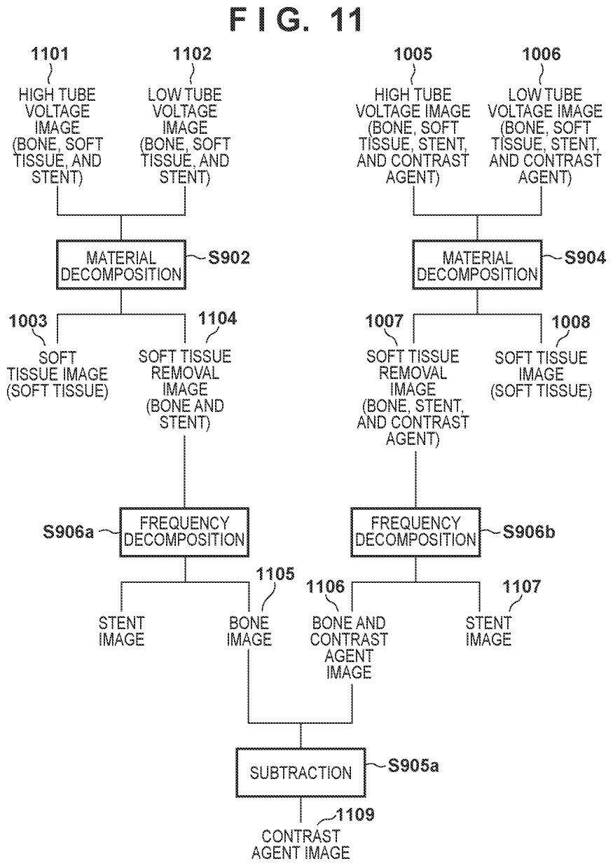

In the method described so far in the fourth embodiment, decomposition into the stent, the contrast agent, the bone, and the soft tissue is performed by using as a mask image an image shot in a state without the stent. However, vascular tracing using a contrast agent is often performed after the stent is placed. Here, if an image that contains the stent is used as a mask image, the stent moves, and therefore the stent in the mask image appears as a reverse image in the image obtained after the subtraction is performed. A method for suppressing such a reverse image will be described below with reference to FIG. 11.

In FIG. 11, a high tube voltage image 1101 and a low tube voltage image 1102 contain a bone, a soft tissue, and a stent. The planar distribution obtaining unit 115 performs material decomposition on the high tube voltage image 1101 and the low tube voltage image 1102 (step S902) and generates a soft tissue image 1003 and a soft tissue removal image 1104. A stent that greatly moves is present in the soft tissue removal image 1104 to be used as a mask image. Therefore, the frequency decomposition unit 116 extracts a bone image 1105 that does not contain the stent from the soft tissue removal image 1104 by performing frequency decomposition processing (step S906a), and uses the thus-obtained image as a mask image. Processing shown in FIG. 11 eliminates the need for obtaining a mask image that contains the bone and the soft tissue (i.e., a mask image that does not contain the stent or the contrast agent).

Target object images obtained by performing material decomposition include a soft tissue image 1008 and a soft tissue removal image 1007, similarly to FIG. 10. The frequency decomposition unit 116 generates a bone-contrast agent image 1106 and a stent image 1107 by performing frequency decomposition on the soft tissue removal image 1007 (step S906b). The output image generation unit 117 generates a contrast agent image 1109 by performing subtraction (step S905a) for the generated bone-contrast agent image 1106 and the bone image 1105. As described above, the stent can be removed from the mask image by applying frequency decomposition. Accordingly, the use of the processing method in FIG. 11 enables decomposition to be accurately performed for all materials without generating an inverted image (reverse image) even when a stent is present in a mask image.

Although decomposition into a stent, a contrast agent, a bone, and a soft tissue is performed in the fourth embodiment, the decomposition targets are not limited thereto. Decomposition targets can include a first substance that appears in a soft tissue removal image and does not move, a second substance that appears in the soft tissue removal image and greatly moves, and a third substance that appears in the soft tissue removal image, greatly moves, and is smaller than the first substance. In the above example, the first substance corresponds to the bone, the second substance corresponds to the contrast agent (contrast vessel), and the third substance corresponds to the stent. For example, four-material decomposition is performed by applying a catheter marker, instead of the stent, as the third substance. Similarly to the stent, the catheter marker is made of metal and therefore appears in the soft tissue removal image, greatly moves up and down in conjunction with the heart movement, and is thinner than a contrast vessel. For this reason, decomposition is enabled by performing processing described in the fourth embodiment. If a fine object, such as a tube made of organic matter, is visibly present in a soft tissue, frequency decomposition may be performed on the soft tissue image.

### Fifth Embodiment

In the first to fourth embodiments, decomposition into a fine structure (e.g., a guide wire, a catheter) and a large structure (e.g., a bone) is performed based on spatial frequency characteristics (i.e., size). However, there are cases where a fine structure also appears in an image of a bone. For example, since calcium density differs between the outside and inside of the bone, a bone thickness image (a bone image in a soft tissue removal image) tends to have higher pixel values (i.e., bone thickness) at the periphery of the bone than in the surrounding area. For this reason, if an image containing a bone is subjected to frequency decomposition, edges of the bone strongly appear in a high-frequency image in some cases. In the fifth embodiment, auxiliary information is used during frequency decomposition. This makes it possible to determine whether an image is of edges of a bone or a guide wire, and improves decomposition accuracy.

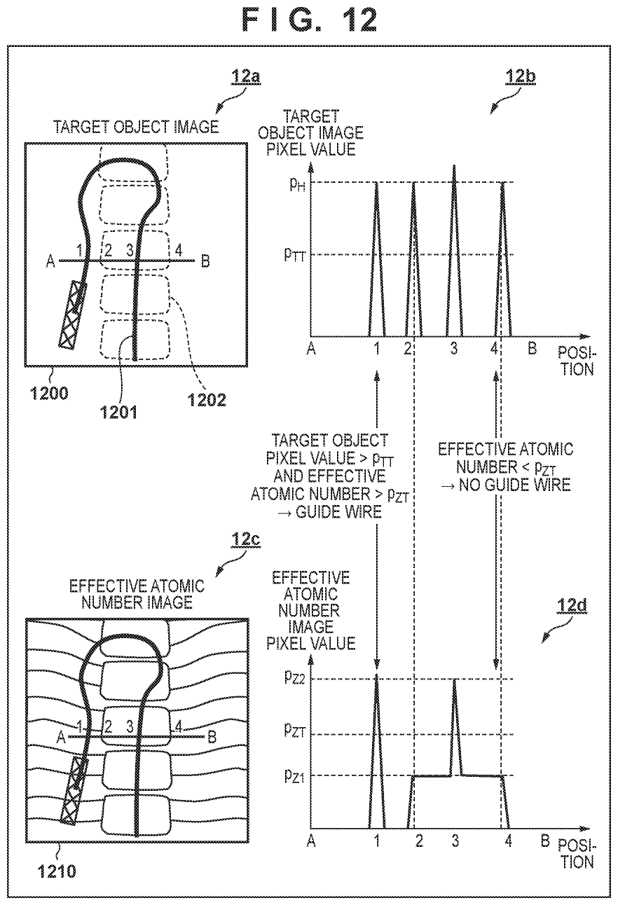

FIG. 12 is a diagram for illustrating processing according to the fifth embodiment. In the fifth embodiment, an effective atomic number image is used as auxiliary information to distinguish bone edges from a stent and a guide wire. An example image 12a is a target object image 1200 (a band limitation image or a combined image of a plurality of band limitation images) obtained by the frequency decomposition unit 116 performing frequency decomposition on a decomposition image obtained by the planar distribution obtaining unit 115. As shown in the example image 12a, not only a guide wire 1201 but also a bone edge 1202 are extracted in the target object image 1200. A profile 12b indicates the results of calculating a profile of the target object image 1200 from position A to position B. The horizontal axis of the profile 12b indicates the position in the target object image 1200. A point denoted as A on the horizontal axis corresponds to position A in the target object image 1200, and a point denoted as B corresponds to position B in the target object image 1200. Numerals 1 to 4 on the horizontal axis in the profile 12b corresponds to numerals 1 to 4 in the example image 12a. Numeral 1 represents the position of a guide wire, numeral 2 represents the position of a bone edge, numeral 3 represents the position of overlapping of the guide wire and the bone, and numeral 4 represents the position of a bone edge. The vertical axis indicates the pixel value of the target object image 1200. In the shown case, the pixel value at the position 1 (guide wire) is substantially equal to the pixel values at the positions 2 and 3 (bone edge) in the target object image. It is therefore difficult to distinguish between the bone edge and the guide wire.

The output image generation unit 117 distinguish between the bone edge and the guide wire using auxiliary information. In the present embodiment, these substances are distinguished by referencing the effective atomic number. An example image 12c shows an effective atomic number image 1210 obtained by means of material identification. A profile 12d is a profile from position A to position B. The positions A and B and numerals 1, 2, 3, and 4 in the example image 12c and the profile 12d are arranged at the same positions as those in the example image 12a and the profile 12b. In general, the higher the effective atomic number of the material of an object is, the larger the effective atomic number image is. Therefore, the effective atomic number image contains information regarding the materials. Accordingly, it is possible to discriminate whether the material of the target object image at each of the positions 1 to 4 is a metal guide wire made of metal or the like, or a bone made of calcium, by referencing the effective atomic number image as auxiliary information.

More specifically, the output image generation unit 117 first assumes, for the target object image 1200, that the position at which the pixel value is above a certain threshold p_{TT} is a candidate for the guide wire (profile 12b). Next, the output image generation unit 117 references the effective atomic number image 1210 and determines that a guide wire is present if the pixel value (effective atomic number) at the same position as the guide wire candidate is not less than the threshold value p_{zt}, and determines that a bone edge is present if this pixel value is less than p_{zt} (profile 12d). This enables accurate determination as to whether a substance appearing in a target object image is a bone edge or a guide wire. The guide wire 1201 can be extracted with high accuracy by performing this determination over the entire target object image.

As described above, according to the first to fifth embodiments, when the human body contains three or more materials, such as a contrast agent, a stent, and a guide wire, in addition to bones and soft tissues when spectral imaging of two-dimensional X-ray images is performed, for example, decomposition into these three or more materials can be enabled with a smaller number of times of imaging. Moreover, in the fourth embodiment, there are cases where the number of times of imaging increases in order to shoot a mask image, but a more accurate decomposition image can be provided.

According to the present disclosure, materials can be separated from a decomposition image obtained by means of spectral imaging or the like, without using a mask image.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An image processing apparatus that processes a radiation image comprising:
a generation unit configured to generate a decomposition image representing a planar distribution related to a material, using a plurality of radiation images of an object containing a target object that correspond to different radiation energies; and
an obtaining unit configured to obtain a target object image related to the target object using a band limitation image corresponding to a frequency band related to a size of the target object, the band limitation image being obtained by performing frequency decomposition on the decomposition image.

2. The image processing apparatus according to claim 1,
wherein the generation unit generates a plurality of decomposition images by performing material decomposition using the plurality of radiation images, and
the generation unit generates the band limitation image by performing frequency decomposition on one decomposition image containing the target object, of the plurality of decomposition images.

3. The image processing apparatus according to claim 1,
wherein the generation unit generates an effective atomic number image or an area density image as the decomposition image by performing material identification using the plurality of radiation images, and
the generation unit generates the band limitation image by performing frequency decomposition on the effective atomic number image or the area density image.

4. The image processing apparatus according to claim 1,
wherein the generation unit performs frequency decomposition on the decomposition image using one of a Laplacian pyramid, Fourier transform, and differentiation.

5. The image processing apparatus according to claim 1,
wherein the obtaining unit obtains the target object image based on at least one of a plurality of band limitation images that are obtained by performing frequency decomposition on the decomposition image and correspond to a plurality of frequency bands.

6. The image processing apparatus according to claim 5,
wherein the obtaining unit obtains the target object image based on the plurality of band limitation images and information regarding the size of the target object.

7. The image processing apparatus according to claim 6,
wherein the obtaining unit selects one of the plurality of band limitation images as the target object image based on the plurality of band limitation images and a frequency corresponding to a size of the target object image on an image, the size being set based on the information regarding the size of the target object.

8. The image processing apparatus according to claim 5,
wherein the obtaining unit selects one of the plurality of band limitation images as the target object image based on an imaging condition of the radiation image.

9. The image processing apparatus according to claim 5,
wherein the obtaining unit obtains a plurality of target object images corresponding to different target objects by weighting the plurality of band limitation images obtained by performing frequency decomposition on the decomposition image in accordance with an imaging condition of the radiation image and combining the plurality of weighted band limitation images, or by using a plurality of sets of weighting coefficients corresponding to the different target objects.

10. The image processing apparatus according to claim 9,
wherein the generation unit generates band limitation images, the number of which corresponds to the number of types of target objects to be extracted, and
the image processing apparatus further comprises a switching unit configured to switch the number of band limitation images.

11. The image processing apparatus according to claim 1,
wherein the target object is one of a guide wire, a stent, a coil, a contrast agent, and a structure made of lime.

12. The image processing apparatus according to claim 1,
wherein the generation unit generates a decomposition image serving as a mask image using two or more radiation images obtained by imaging the object in a state of not containing the target object, with the radiation of different levels of energy,
the image processing apparatus further comprises a subtraction unit configured to subtract the mask image from the decomposition image, and
the generation unit generates a band limitation image by performing frequency decomposition on the decomposition image after being subjected to the subtraction performed by the subtraction unit.

13. The image processing apparatus according to claim 1,
wherein the generation unit generates a decomposition image serving as a mask image using two or more radiation images obtained by imaging the object that does not contain the target object, with the radiation of different levels of energy, and
the image processing apparatus further comprises a subtraction unit configured to subtract, from the band limitation image, a mask band limitation image obtained by performing frequency decomposition on the mask image.

14. The image processing apparatus according to claim 1,
wherein the obtaining unit extracts the target object from the target object image obtained based on the band limitation image, by further using auxiliary information.

15. The image processing apparatus according to claim 14,
wherein the auxiliary information is a pixel value of an effective atomic number image.

16. An image processing method for processing a radiation image comprising:
generating a decomposition image representing a planar distribution related to a material, using a plurality of radiation images of an object containing a target object that correspond to different radiation energies; and
obtaining a target object image related to the target object using a band limitation image corresponding to a frequency band related to a size of the target object, the band limitation image being obtained by performing frequency decomposition on the decomposition image.

17. A non-transitory computer-readable storage medium storing a program for causing a computer to execute the image processing method according to claim 16.
